(19) United States
(12) Patent Application Publication
(10) Pub. No.: US 2026/0284366 A1
(43) Pub. Date: Sep. 24, 2026

(54) PLIABLE GUIDEWIRE AND METHOD OF MANUFACTURE

(71) Applicant: Merit Medical Systems, Inc., South Jordan, UT (US)

(72) Inventors: Ryan Davis, Centerville, UT (US); Faraz Ul Hassan, South Jordan, UT (US); Tomasz Hawrylak, Oranhill (IE); R.B. Hayes, Salt Lake City, UT (US); Emma Keady, Kinvara (IE)

(21) Appl. No.: 19/572,619

(22) Filed: Mar. 19, 2026

Related U.S. Application Data
(60) Provisional application No. 63/775,501, filed on Mar. 21, 2025.

(51) Int. Cl. A61M 25/09

(52) U.S. Cl. CPC A61M 25/09; A61M 2025/09083; A61M 2025/09133; A61M 2025/09166; A61M 2205/0222; A61M 2205/0238

(57) ABSTRACT

A guidewire is provided. The guidewire can include a core wire and a pliable hypotube coupled to the core wire. The pliable hypotube can include a plurality of cuts disposed in a helical pattern.

### RELATED CASES

This application claims priority to United States Provisional Application No. 63/775,501, filed on Mar 21, 2025 and titled “Pliable Guidewire and Method of Manufacture,” which is hereby incorporated by reference in its entirety.

### TECHNICAL FIELD

The present disclosure relates generally to medical devices. More specifically, the present disclosure relates to guidewire devices, including micro-guidewires.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a lateral view of a pliable guidewire, according to embodiments disclosed herein.

FIG. 2 illustrates an exploded view of the pliable guidewire of FIG. 1, according to embodiments disclosed herein.

FIG. 3 illustrates a lateral view of a core wire of the pliable guidewire of FIG. 1, according to embodiments described herein.

FIG. 4 illustrates a lateral view of a hypotube of the pliable guidewire of FIG. 1, according to embodiments described herein.

FIG. 5 illustrates a lateral, cutaway view of the hypotube of the pliable guidewire of FIG. 1, according to embodiments described herein.

FIG. 6 illustrates a lateral view of a radiopaque wire of the pliable guidewire of FIG. 1, according to embodiments described herein.

FIG. 7 illustrates a lateral, cutaway view of a distal end of the pliable guidewire of FIG. 1, according to embodiments described herein.

FIG. 8 illustrates an anterior view of the distal end of the pliable guidewire of FIG. 1, according to embodiments described herein.

### DETAILED DESCRIPTION

Medical guidewires often include a pliable tip, or end region. Flexibility at a distal region allows a guidewire to bend or adapt to curves and branches of blood vessels, particularly in cases where anatomy is challenging. A pliable, atraumatic tip often provides the agility to maneuver through narrow or winding vessels, enhancing the overall effectiveness and safety of the procedure.

Often, a guidewire will include a region with varying flexibilities e.g., along its length or tip, to facilitate navigation of the vasculature. For example, increased rigidity in a proximal part of a guidewire can offer stability and pushability. Increased flexibility, particularly at the distal region or tip can be used to navigate through tight curves and branches without causing trauma to the vessel walls. A gradient or gradual increase in flexibility can thus enhance a guidewire’s ability to maneuver through complex and tortuous pathways.

Other features of medical guidewires can have an impact on performance and safety. For example, torque response, or torsional rigidity, allows for control and maneuverability within the vascular system. Hydrophilic properties reduce friction and facilitate smooth navigation through vessels. Additionally, visibility under fluoroscopy enables clinicians to accurately track the guidewire's position.

Conventional guidewires attempt to offer these features through a combination of tapered design and cutting, welding, and shaping techniques. For instance, conventional guidewires can offer a tapered design that gradually decreases the diameter of the guidewire from the proximal end to the distal tip.

Certain pliable guidewires, and their methods of manufacture, may have some limitations. For instance, the design and construction of varying materials, and precise transitions between rigid and pliable sections, can be difficult to consistently manufacture. This constraint can result in high production costs during manufacturing. Material limitations can also create complexities. For example, metals such as stainless steel or nitinol, which offer desirable properties such as biocompatibility, can be difficult to shape or bond.

Systems and methods of the present disclosure address the above and other challenges by providing systems and methods related to a pliable guidewire and a corresponding method of manufacture. In some embodiments, a pliable guidewire can be manufactured with a plurality of helical cuts. The series of helical cuts can increase in spacing and/or size, from a distal end of the guidewire towards the proximal end of the guidewire. This can allow for a guidewire with decreasing flexibility along the proximal direction, towards the user. In some embodiments, this can allow for enhanced pushability, while maintaining torsional rigidity, and flexibility at the distal end.

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive).

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

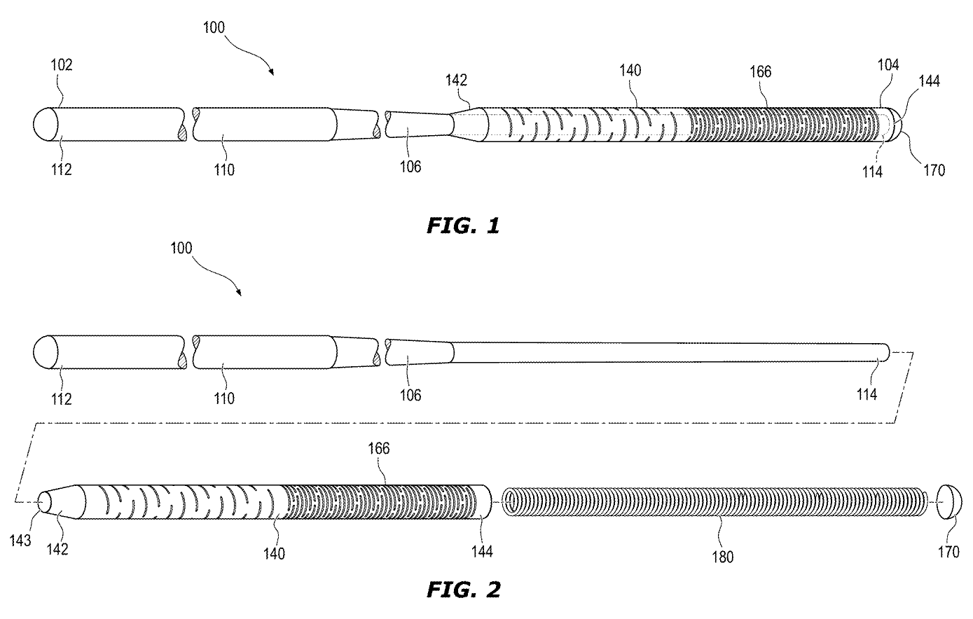

FIG. 1 illustrates a lateral view of a pliable guidewire 100, according to embodiments disclosed herein. In the illustrated embodiment, pliable guidewire 100 includes a proximal end 102, a distal end 104, and a middle portion 106. A core wire 110 extends a longitudinal length of the pliable guidewire 100. A hypotube 140 is disposed around a circumference of a distal portion of core wire 110.

Proximal end 102 of pliable guidewire 100 can include, or be a part of, core wire 110. Core wire 110 can extend through a core, or central axis of pliable guidewire 100. In some embodiments, proximal end 102 can be configured to be manipulated by a user of the device. For instance, proximal end 102 of core wire 110 can be configured to be manipulated by a user of the device to navigate a vascular system (e.g., during a medical procedure).

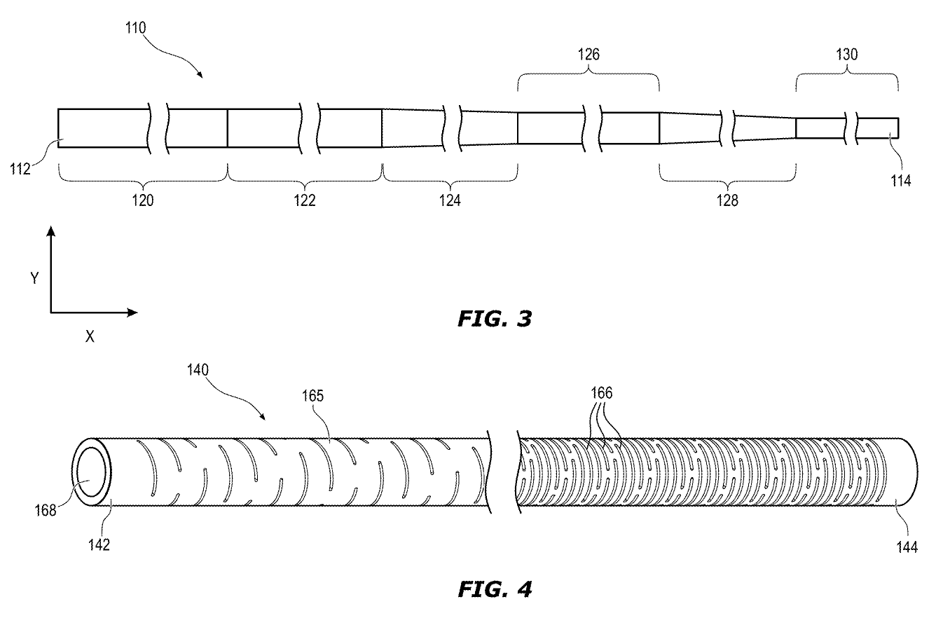

In some embodiments, core wire 110 can be configured to progressively increase in pliability, as core wire 110 extends in the distal direction. Otherwise stated, a pliability of core wire 110 can increase along the length of core wire 110, from a proximal end 112 to a distal end 114. As will be further discussed with respect to FIG. 3, core wire 110 may be tapered in the direction of, or traversing from, proximal end 112 to distal end 114 of core wire 110. In contrast, hypotube 140 may be devoid of a taper, in some embodiments.

Hypotube 140 is disposed at a distal region of the pliable guidewire 100. Hypotube 140 can be disposed exteriorly around a circumference of a portion of core wire 110.

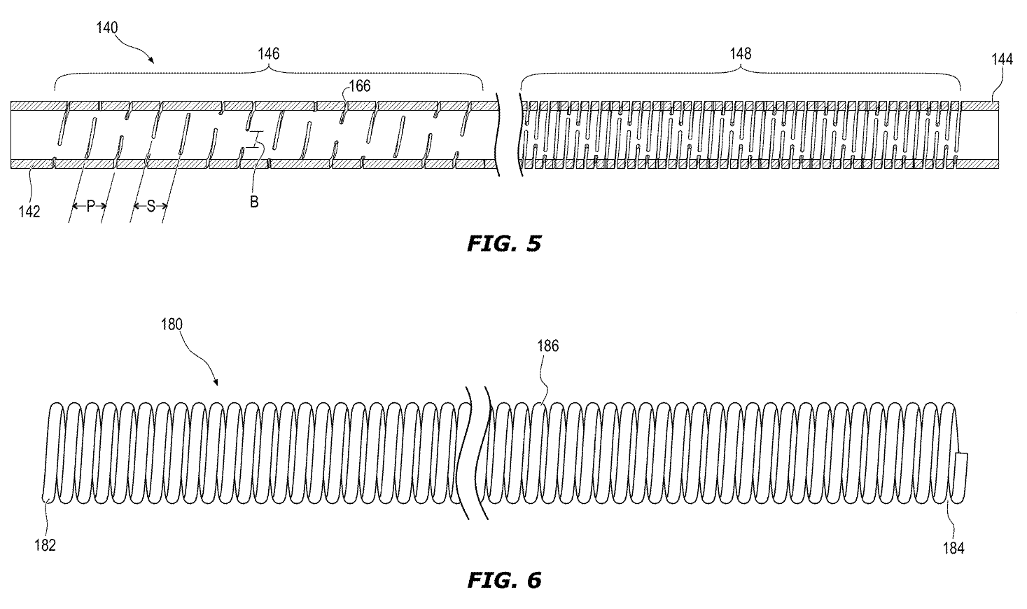

In some embodiments, hypotube 140 can be configured to progressively increase in pliability, as hypotube 140 extends in the distal direction. Otherwise stated, a pliability of hypotube 140 can increase along the length of hypotube 140, from a proximal end 142 to a distal end 144. Slits 166 (e.g., cuts, slots, gaps, or openings) disposed on an exterior surface of hypotube 140, or through hypotube 140, can be used to create pliability in a controlled manner as will be further discussed with respect to FIG. 5. Slits 166 can be formed or machined into pliable guidewire 100 along a longitudinal length of hypotube 140. In some embodiments, slits 166 can be formed by machining (e.g., via saw-cutting, waterjet cutting, electrical discharge machining, etc.), through chemical processes (e.g., chemicals etching), laser cutting, etc.

Pliable guidewire 100 (e.g., core wire 110, hypotube 140, and/or end cap 170) can be constructed from material including nickel titanium alloy (e.g., Nitinol), stainless steel, or other biocompatible materials.

FIG. 2 illustrates an exploded view of pliable guidewire 100 of FIG. 1, according to embodiments disclosed herein. As seen in the illustrated embodiment, core wire 110 includes proximal end 112 and distal end 114. Hypotube 140 includes proximal end 142 and distal end 144.

As will be further seen, in some embodiments, pliable guidewire 100 can include a radiopaque wire 180 coiled around a circumference of core wire 110. Radiopaque wire 180 can provide enhanced visibility for distal end 104 of pliable guidewire 100 (e.g., under fluoroscopic imaging).

Proximal end 142 of hypotube 140 can include an aperture 143 for mating or coupling with the core wire 110. For example, in some embodiments, hypotube 140 and core wire 110 can be bonded via an adhesive placed at or near aperture 143.

In some cases, core wire 110, radiopaque wire 180, and hypotube 140 can be joined at distal end 104 of pliable guidewire 100. For instance, in some cases, core wire 110, radiopaque wire 180, and hypotube 140 can be joined at end cap 170. For instance, end cap 170 can translationally and rotationally fix core wire 110, radiopaque wire 180, and hypotube 140 relative to each other.

Radiopaque wire 180 can be formed from may be gold, platinum alloy, gadolinium, dysprosium, tungsten, compounds thereof, etc.

Distal end 104, hypotube 140, or any part of pliable guidewire 100 can be smoothened, for instance, to reduce friction or resistance to movement within vasculature. For example, in some embodiments, the surface of pliable guidewire 100 can be electropolished. In some embodiments a hydrophilic coating may be applied to pliable guidewire 100 and/or components such as core wire 110, hypotube 140, and/or radiopaque wire 180. For instance, in some cases, a coating such as a silicone-based oil or polymer can be applied.

In some embodiments, a hydrophilic coating can be applied to hypotube 140 such that the coating permeates into slits 166. In some embodiments the coating can permeate into slits 166, without saturating the space between slits or the space within hypotube 140. In other words, the hydrophilic coating is not disposed between core wire 110 and hypotube 140.

FIG. 3 illustrates a lateral view of core wire 110 of pliable guidewire 100 of FIG. 1, according to embodiments described herein. In the illustrated embodiment, core wire 110 includes several zones or sections of varying diameter and longitudinal distance. As seen in the illustrated embodiment, core wire 110 includes sections 120, 122, 124, 126, 128, and 130. In alternate embodiments, core wire 110 can include more or fewer sections. In the illustrated embodiment, section 120 is the proximal-most section of core wire 110. Section 130 is the distal-most section of core wire 110. In some embodiments, distal end 114 may be flattened. In some embodiments, section 130 may be flattened.

As seen in the illustrated embodiment, core wire 110 includes 6 sections. In alternate embodiments, core wire 110 can include more or fewer sections. In some cases, sections of core wire 110 can vary in length, in others, the sections can be uniform in length. For example, in some embodiments, each section of the core wire 110 can be anywhere between 20 millimeters and 3,000 millimeters in length. Core wire 110 can be of an overall length of anywhere between 1,400 millimeters and 3,600 millimeters.

In some embodiments, core wire 110 can be formed of stainless steel, nitinol, or any other biocompatible material. A taper can be machined (e.g., via a grinding process) onto core wire 110.

In some examples, core wire 110 can include a coating (e.g., a hydrophobic coating, a hydrophilic coating, a lubricious coating) for some, or all, or any configuration of the sections. For example, in some embodiments, section 120 can include a PTFE coating, while the other sections can remain uncoated.

An outer diameter of core wire 110 can taper as the distal end 114 is approached. Otherwise stated, in some, or all of the zones or sections illustrated within FIG. 3, the outer diameter of core wire 110 can vary, or decrease (in some cases, the outer diameter across one or more sections can remain constant). For example, in some embodiments, the outer diameter of the core wire 110 can decrease from about 0.018 inches (e.g., at the proximal end 112) to about 0.0020 inches (e.g., at the distal end 114). In some cases, such a tapering, or reduction in diameter can be evenly distributed throughout sections of the core wire 110. Alternatively, some sections may include more aggressive tapering. Accordingly, any section or zone of the core wire 110 may include up to 90% of the overall tapering, or reduction in outer diameter, of the core wire 110.

FIG. 4 illustrates a perspective view of hypotube 140 of pliable guidewire 100 of FIG. 1, according to embodiments described herein. In the illustrated embodiment, hypotube 140 includes proximal end 142 and distal end 144. A series of cuts or slits 166 can be seen extending from exterior wall 165 of hypotube 140, to inner lumen 168 of hypotube 140. As illustrated, hypotube 140 may comprise a proximal section 146 and a distal section 148 that may have different characteristics for slits 166. For ease of illustrating and discussing the different characteristics of slits 166, hypotube 140 includes break lines to distinguish proximal section 146 and distal section 148. Hypotube 140 can extend anywhere between 20 centimeters to 40 centimeters (e.g., from distal end 144 to proximal end 142).

Slits 166 can be angled or arranged in a helical pattern or fashion. In other words, slits 166 are not orthogonal to an axis of hypotube 140. The decreasing distance of slits 166 can allow for greater flexibility. Otherwise stated, the distal end 144 of hypotube 140 can be more flexible than proximal end 142.

As will be further discussed with respect to FIG. 5, slits 166 can be configured in sections, with varying helical (e.g., both longitudinal and circumferential) spacing. Otherwise stated, as the helical distance between individual slits of slits 166 is increased, a longitudinal length between slits can increase, as well as a circumferential distance that is perpendicular to a longitudinal length.

Slits 166 can further be configured to prevent or stop over-bending of the hypotube 140 and/or pliable guidewire 100. For example, when pliable guidewire 100 is flexed, or bent, slits 166 and the space therein can close, such that side-walls of the slits contact or stop a bend.

FIG. 5 illustrates a lateral, cutaway view of hypotube 140 of pliable guidewire 100 of FIG. 4, according to embodiments described herein. As seen in the illustrated embodiment, hypotube 140 includes proximal end 142 and distal end 144 with a proximal section 146 and distal section 148 divided by break lines. A series of cuts or slits 166 can be seen extending along and/or throughout hypotube 140. Each slit of slits 166 can be of a width (e.g., a laser kerf) of anywhere between 0.0005 inches and 0.0010 inches. As discussed above, slits 166 can be formed by machining (e.g., via saw-cutting, waterjet cutting, electrical discharge machining, etc.), through chemical processes (e.g., chemicals etching), laser cutting, etc. Slits 166 may be cut in a clockwise direction from distal end 144. In some embodiments, slits 166 may be cut in a counterclockwise direction from distal end 144.

In the illustrated embodiment, hypotube 140 includes several zones or sections where slits 166 are of varying pitch (P), bridge distance (B), and/or strut width (S). Pitch (P) refers to the distance (along the longitudinal axis) between corresponding slits of a complete turn of the helix (as seen in FIG. 5). Bridge distance (B) refers to the helical distance between two consecutive slits of slits 166. The helical “filled-in” space between consecutive slits is referred to as a bridge. Strut width (S) can be defined as the material separating longitudinally consecutive slits. A strut width (S) or thickness can accordingly be contingent on the pitch (P) of the helical pattern.

In some embodiments, hypotube 140 may include multiple distinct sections with proximal section 146 and distal section 148. For example, distal section 148 may comprise four distinct sections with different pitch (P), bridge distance (B), and/or strut width (S). Proximal section 146 may comprise six distinct sections with different pitch (P), bridge distance (B), and strut width (S). In alternate embodiments, hypotube 140 can include more or fewer sections.

In some cases, proximal section 146 and distal section 148 of hypotube 140 can vary in length, in others, the sections can be uniform in length. For example, in some embodiments, each or any of the distinct sections within proximal section 146 and distal section 148 of hypotube 140 can be anywhere between 10 millimeters and 70 millimeters in length and the overall length of hypotube 140 may be 350 mm.

In some embodiments, pitch and bridge distance can vary with respect to any section (e.g., pitch and bridge distance can gradually increase, moving in the proximal direction). In some cases, pitch (P), bridge distance (B), and/or strut width (S) can be similar or be constant within one or more sections. For example, in some embodiments, the pitch (P) of the helical pattern of slits 166 within FIG. 5 can be or vary anywhere from 0.0010 inches to 0.0080 inches. For example, in some embodiments, the pitch (P) of the helical pattern nearer distal end 144 can be closer to 0.0010 inches, and gradually increase from distal end 144 to proximal end 142. Closer to proximal end 142 the pitch (P) of the helical pattern can be closer to 0.0080 inches. In some embodiments, the pitch (P) increases within each section of hypotube 140 along a longitudinal length of hypotube 140. In correlation with the pitch (P), an angle of any slit of slits 166 of the hypotube 140 can vary from between 0 degrees (or 0.00 radians) to 13 degrees (or 0.23 radians) (e.g., from normal to the longitudinal axis), including from 4 degrees (or 0.07 radians) to 13 degrees (or 0.23 radians).

In some embodiments, the patterns of cuts or slits 166 in the hypotube 140 may be such that the slits are disposed perpendicular (e.g, at a 0 degree angle) over a segment of the hypotube 140, then transition to a helical pattern with a different pitch. The segment with perpendicular slits may have a lower stiffness as compared to segments with a helical pattern. In some instances a combination of one or more segments with perpendicular slits 166 and one or more segments with helically disposed slits 166 (including segments with a constant pitch and/or segments having one or more pitches angles and/or portions with a pitch that continuously varies along a length) can be utilized to tune or control flexibility and/or facilitate manufacturing.

In addition, in some embodiments, the bridge length (e.g., a helical length) of the series of bridges between the (series of) slits 166 can be anywhere from 0.0010 inches and 0.0060 inches. For example, in some embodiments, bridge distance (B) of the helical pattern nearer distal end 144 can be closer to 0.0010 inches, and gradually increase from distal end 144 to proximal end 142. Closer to proximal end 142 the bridge distance (B) of the helical pattern can be closer to 0.0060 inches. In some embodiments, the bridge distance (B) in distal section 148 may increase from distal end 144 to proximal section 146. In some embodiments, the bridge distance (B) in proximal section 146 may be constant along the entire length of proximal section 146. Further, longitudinally adjacent bridges may be longitudinally offset from each other.

In some cases, slits 166 (e.g., a first/final slit of slits 166) can begin at a longitudinal distance anywhere between 0.0080 inches and 0.0090 inches from distal end 144 of hypotube 140. In some cases, slits 166 (e.g., a first/final slit of slits 166) can end at longitudinal distance anywhere from 0.0090 inches to 0.0110 inches from the proximal end 142 of hypotube 140.

In some cases, slits 166 can be set in a helical pattern such that there is anywhere between 1.5 helical cuts per rotation around the circumference of the hypotube to 3 helical cuts per rotation around the circumference, including from 2 helical cuts per rotation around the circumference of the hypotube 140 and 3 helical cuts per rotation around the circumference of the hypotube 140. In some embodiments, the cut per rotation may be about 2.5. For example, in some embodiments, each cut per rotation (e.g., 360 degrees) around the circumference of the hypotube 140 can span anywhere from 100 degrees to 200 degrees, including from 100 degrees to 140 degrees. In some embodiments, each bridge per rotation (e.g., 360 degrees) around the circumference of the hypotube 140 can span anywhere from 10 degrees to 45 degrees.

In some embodiments, the helical cuts per rotation is constant along the longitudinal length of hypotube 140. Accordingly, as the bridges distance (B) increases, the length of each slit 166 must decrease to maintain the constant helical cut per rotation ratio of hypotube 140. In some embodiments, strut width (S) can have a thickness of anywhere between 0.0010 inches and 0.0075 inches. In some embodiments, the strut width (S) can increase in every section of hypotube 140 along the entire longitudinal length of hypotube 140.

As discussed above, proximal section 146 and distal section 148 may have different characteristics. For example, in some instances, the wire may be designed with maximum flexibility at the distal end while also comprising areas of lower and higher amounts of flexibility anywhere along the length of the hypotube 140. Further, the zone of maximum flexibility may or may not be at the distal most end. For instance, embodiments wherein a highly flexible segment is desired at some point longitudinally offset from the distal tip then the features of the hypotube 140 (e.g, pitch, bridge, angles, etc.) could be modified to create those characteristics. Furthermore, the features of the hypotube 140 can be configured to create desired zones of flexibility anywhere along the length of the hypotube 140. For instances, wires with zones of flexibility configured to interact or traverse particular anatomical features can have flexible segments tuned for particular applications.

In some embodiments, proximal section 146 may comprise multiple distinct sections with different characteristics and distal section 148 may comprise multiple distinct sections with different characteristics. For example, in distal section 148, the strut width (S) may increase from distal end 144 to proximal section 146, the pitch (P) may increase from distal end 144 to proximal section 146, the bridge distance (B) may increase from distal end 144 to proximal section 146, the length of slit 166 may decrease from distal end 144 to proximal section 146, and cuts per rotation may be constant from distal end 144 to proximal section 146. Further, each characteristic may only increase or decrease when transitioning from adjacent distinct sections of distal section 148.

In some embodiments, in proximal section 146, the strut width (S) may increase from distal section 148 to proximal end 142, the pitch (P) may increase from distal section 148 to proximal end 142, the bridge distance (B) may be constant from distal section 148 to proximal end 142, the length of slit 166 may be constant from distal section 148 to proximal end 142, and cuts per rotation may be constant from distal section 148 to proximal end 142. Further, each characteristic may only increase or decrease when transitioning from adjacent distinct sections of proximal section 146.

Additionally, the bridge position (relative to adjacent bridges/struts) may also be adjusted to tune or control characteristics of the hypotube. For example, the bridge may be located at the midpoint of the adjacent struts, which may reduce stiffness, as compared to configurations where the bridges are closer to adjacent bridges (including embodiments where the bridges are stacked and/or aligned).

In some examples, hypotube 140 can include a coating (e.g., a hydrophilic or lubricious coating) for some, or all, or any configuration of the sections or slits 166. For instance, in some embodiments, the coating may penetrate the slits and/or the interior of hypotube 140. In some embodiments, the coating does not penetrate the interior of hypotube 140. Alternatively, the coating may only be on one or more sections of the outer circumferential surface.

In some cases, a hydrophilic or lubricious coating can penetrate some slits of slits 166 (e.g., within a single, or multiple, sections of the hypotube 140), and not others. Additionally, with respect to specific slits of slits 166, the hydrophilic or lubricious coating can penetrate a portion of, or only partially penetrate, specific slits of slits 166. In some embodiments, the hydrophilic or lubricious coating can penetrate all of the slits of slits 166.

FIG. 6 illustrates a lateral view of radiopaque wire 180 of pliable guidewire 100 of FIG. 1 to FIG. 2, according to embodiments described herein. As seen in the illustrated embodiment, radiopaque wire 180 includes a proximal end 182 and a distal end 184. A midbody portion 186 of radiopaque wire 180 can include coils that are wound with a specific pitch.

In some cases, radiopaque wire 180 can be anywhere between 5 centimeters and 25 centimeters in length. A pitch of the coil of the radiopaque wire 180 can be anywhere between .00100 inches and .00200 inches. In some cases, the pitch of radiopaque wire 180 may vary over a longitudinal length of radiopaque wire 180. In other cases, the pitch of radiopaque wire 180 may be constant over the longitudinal length of radiopaque wire 180.

Radiopaque wire 180 can include a wire with a circular, oval, or ribbon-like cross-section. In some embodiments, radiopaque wire 180 may have an oval cross-sectional shape with a major axis of the oval cross-sectional shape that is perpendicular to the axis of core wire 110.

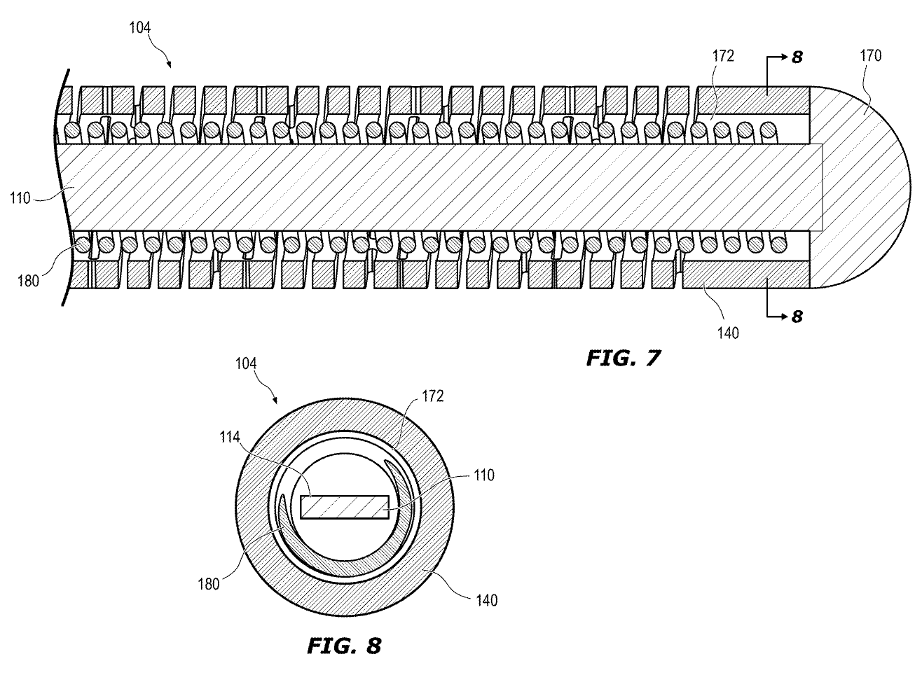

FIG. 7 illustrates a cutaway, lateral view of distal end 104 of pliable guidewire 100 of FIG. 1, according to embodiments described herein. As seen in the illustrated embodiment, distal end 104 of pliable guidewire 100 includes core wire 110, radiopaque wire 180, and hypotube 140.

Core wire 110, radiopaque wire 180, and hypotube 140 can be attached or joinedly fixed to or at end cap 170. For example, in some embodiments, core wire 110, radiopaque wire 180, and hypotube 140 can be fixed to end cap 170 via an adhesive. End cap 170 may be fabricated from a polymer and have a semi-spherical shape. End cap 170 is distal end 104 of pliable guidewire 100 and therefore the semi-spherical shape gives distal end 104 of pliable guidewire 100 an atraumatic shape. Other potential shapes of end cap 170 are within the scope of this disclosure that also provide atraumatic shapes.

As seen, a void (e.g., void 172) or space can exist between the radiopaque wire 180 and the internal circumferential surface of the hypotube 140. In some embodiments, the portion of pliable guidewire 100 seen in FIG. 7 can be coated with a hydrophilic or lubricious coating. In some cases, the hydrophilic or lubricious coating can extend into the slits 166, but not extend into the interior space of hypotube 140 (e.g., void 172). In alternate cases, the hydrophilic or lubricious coating may extend throughout the interior space of hypotube 140 (e.g., into void 172).

FIG. 8 illustrates an anterior view of distal end 104 of pliable guidewire 100 of FIG. 1 to FIG. 2, according to embodiments described herein. As seen in the illustrated embodiment, radiopaque wire 180 is disposed within hypotube 140. Core wire 110 is within radiopaque wire 180.

As seen in FIG. 8, in some cases, a distal end 114 of the core wire 110 can be flattened, or rectangular (e.g., as opposed to circular, which distal end 114 may be in some embodiments). A flattened distal end 114 can increase flexibility, tip shape retention, or pliability, at distal end 104 of the pliable guidewire 100.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A guidewire comprising:
a core wire; and
a pliable hypotube coupled to a distal portion of the core wire and disposed around the distal portion of the core wire,
wherein the pliable hypotube comprises a plurality of cuts disposed in a helical pattern.

2. The guidewire of claim 1, wherein the helical pattern increases in pitch from a distal end of the pliable hypotube to a proximal end of the pliable hypotube.

3. The guidewire of claim 1, wherein the helical pattern comprises a distal end comprising a pitch of about 0.0010 inches; and
wherein the helical pattern comprises a proximal end comprising a pitch of about 0.0080 inches.

4. The guidewire of claim 1, wherein the helical pattern comprises between 2 and 3 cuts per rotation.

5. The guidewire of claim 1, wherein cuts per rotation of the helical pattern is constant along a length of the pliable hypotube.

6. The guidewire of claim 1, wherein the helical pattern comprises a distal end comprising a cut of the plurality of cuts extending about 0.07 radians; and
wherein the helical pattern comprises a proximal end comprising a cut of the plurality of cuts extending 0.23 radians.

7. The guidewire of claim 1, further comprising a radiopaque wire coiled around the core wire and disposed within the pliable hypotube,
wherein the radiopaque wire comprises a circular cross-sectional shape.

8. The guidewire of claim 1, wherein the pliable hypotube comprises a hydrophilic coating disposed on an exterior circumferential surface of the pliable hypotube.

9. The guidewire of claim 8, wherein the hydrophilic coating extends into at least a portion of an internal surface of the plurality of cuts.

10. The guidewire of claim 8, wherein the hydrophilic coating is not disposed between the core wire and the pliable hypotube.

11. The guidewire of claim 1, further comprising a plurality of bridges, wherein each bridge of the plurality of bridges is disposed between each set of consecutive cuts of the plurality of cuts.

12. The guidewire of claim 1, wherein the pliable hypotube further comprises a plurality of struts, wherein each strut of the plurality of struts is disposed longitudinally between each set of longitudinally consecutive cuts of the plurality of cuts; and
wherein a longitudinal length of each strut of the plurality of struts increases from a distal end of the pliable hypotube to a proximal end of the pliable hypotube.

13. A guidewire comprising:
a core wire; and
a pliable hypotube coupled to a distal portion of the core wire and disposed around the distal portion of the core wire, the pliable hypotube comprising:
a plurality of cuts disposed in a helical pattern; and
a plurality of bridges, wherein each bridge of the plurality of bridges is disposed between each set of consecutive cuts of the plurality of cuts.

14. The guidewire of claim 13, wherein each bridge of the plurality of bridges is disposed helically between each set of helically consecutive cuts of the plurality of cuts.

15. The guidewire of claim 13, wherein a helical length of each bridge of the plurality of bridges increases from a distal end of the pliable hypotube to a proximal end of the pliable hypotube.

16. The guidewire of claim 13, wherein the pliable hypotube comprises a distal portion and a proximal portion;
wherein a helical length of each bridge of the plurality of bridges in the distal portion of the pliable hypotube increases from a distal end to the proximal portion;
wherein the helical length of each bridge of the plurality of bridges in the proximal portion is the same, and
wherein the plurality of bridges comprises bridges of a helical length between about 0.0010 inches and 0.0060 inches.

17. The guidewire of claim 13, wherein the pliable hypotube further comprises a plurality of struts, wherein each strut of the plurality of struts is disposed longitudinally between each set of longitudinally consecutive cuts of the plurality of cuts, and
wherein the plurality of struts comprises struts of a longitudinal length between about 0.0010 inches and 0.0075 inches.

18. The guidewire of claim 17, wherein a longitudinal length of each strut of the plurality of struts increases from a distal end of the pliable hypotube to a proximal end of the pliable hypotube.

19. The guidewire of claim 17, wherein longitudinally adjacent bridges of the plurality of bridges are longitudinally offset.

20. The guidewire of claim 13, wherein the helical pattern increases in pitch from a distal end of the pliable hypotube to a proximal end of the pliable hypotube.
